(19) United States
(12) Patent Application Publication
(10) Pub. No.: US 2022/0047559 A1
(43) Pub. Date: Feb. 17, 2022

(54) DRUG COMPOSITION FOR TREATING BREAST CANCER AND METHOD FOR MANUFACTURING THE SAME

(71) Applicant: National Chiao Tung University, Hsinchu City (TW)

(72) Inventors: Dean-Mo Liu, Baoshan Township (TW); Chu-Ting Li, Tainan City (TW); Wen-Lien Wang, Taichung City (TW)

(73) Assignee: National Chiao Tung University, Hsinchu City, TAIWAN

(21) Appl. No.: 17/066,087

(22) Filed: Oct. 8, 2020

(30) Foreign Application Priority Data
Aug. 11, 2020 (TW) 109127151

(51) Int. Cl. A61K 31/4196; A61K 31/12; A61K 47/68; A61K 9/48

(52) U.S. Cl. CPC A61K 31/4196; A61K 9/4816; A61K 47/6855; A61K 31/12

(57) ABSTRACT

The present invention relates to a drug composition and method for treating breast cancer, and more specifically, to use carboxymethyl-hexanoyl chitosan (CHC) to co-encapsulate a heat shock protein 90 (HSP90) inhibitor and a hydrophobic drug. The two drugs can be co-encapsulated with high encapsulation efficiency and co-delivered to breast cancerous cells, and achieve a synergistic efficacy to kill the cancerous cells.

This application claims priority of Application No. 109127151 filed in Taiwan on 11 Aug. 2020 under 35 U.S.C. § 119; the entire contents of all of which are hereby incorporated by reference.

### BACKGROUND OF THE INVENTION

### Field of the Invention

The present invention relates to a drug composition for treating breast cancer and a method for manufacturing the same, particularly to a drug composition wherein a combination of drugs is encapsulated in a nanocarrier for treating breast cancer and a method for manufacturing the same.

### Description of the Related Art

Breast cancer is the most common cancer in females, ranked No. 2 (behind lung cancer) among the cancer-related mortalities of females. The breast cancer-induced death is primarily due to metastasis. The existing therapeutic methods have significantly increased the lifetime of breast cancer patients. However, there are still 30-40% patients dying of recurrence of breast cancer. Breast cancer is a complicated disease, involving different pathological features and clinical syndromes. More and more evidences indicate that the breast cancer correlating with metabolisms having different histopathological features and different biological features should be treated in different therapeutic strategies. Therefore, it is very important for breast cancer treatment to correctly classify breast cancer into subtypes.

Breast cancer may be pathologically classified into two main subtypes: the breast ductal carcinoma (about 90%) and breast lobular carcinoma (about 5%). Other pathological subtypes are seldom seen. However, the most important classification of breast cancer is performed on the cancer cells according to the biological characteristics. The classification is based on the gene expressions of breast cancer cells to obtain the following five subtypes: luminal A breast cancer, luminal B breast cancer, HER2 over-expression breast cancer, basal breast cancer, and normal-like breast cancer.

The HER2 over-expression subtype is about 20-30% in breast cancer, having high recurrence rate and high mortality. The metastasis of the HER2 over-expression subtype is primarily treated with the combination of chemotherapy and targeting therapy. However, multidrug resistance (MDR) is the most significant barrier to chemotherapy. Therefore, the related fields desire to have a breast cancer drug able to lower the action of MDR.

### SUMMARY OF THE INVENTION

One objective of the present invention is to use self-assembly nanocarrier to encapsulate a plurality of drugs having different performances so as to decrease the concentration of drugs, lower the side-effects of drugs, and co-deliver the drugs to breast cancer cells, whereby to achieve a synergistic efficacy and kill breast cancer cells.

In order to achieve the abovementioned objective, the present invention provides a drug composition for treating breast cancer, which comprises a nanocarrier, which is assembled with Carboxymethyl-Hexanoyl Chitosan (CHC); at least one heat shock protein 90 (HSP90) inhibitor; and at least one hydrophobic drug, wherein the HSP90 inhibitor and the hydrophobic drug are encapsulated inside the nanocarrier; the HSP 90 inhibitor includes ganetespib; the hydrophobic drug includes curcumin.

In one embodiment, the drug composition is in form of a plurality of particles whose diameters are within a range of 200-500 nm.

In one embodiment, a targeting material is connected to the surface of the drug composition.

In one embodiment, the targeting material includes a monoclonal antibody Trastuzumab.

In one embodiment, the breast cancer is a breast cancer of the HER2 overexpression subtype.

The present invention also provides a method for manufacturing a drug composition for treating breast cancer, which comprises steps: dispersing Carboxymethyl-Hexanoyl Chitosan (CHC), at least one heat shock protein 90 (HSP90) inhibitor and at least one hydrophobic drug in a solvent to form a mixture solution; placing the mixture solution at a lower temperature and agitating the mixture solution for 20-24 hours to form the drug composition, wherein the HSP90 inhibitor and the hydrophobic drug are encapsulated inside the nanocarrier formed via assembly of CHC; the HSP 90 inhibitor includes ganetespib; the hydrophobic drug includes curcumin.

In one embodiment, the ratio of the concentration of ganetespib to the concentration of curcumin is 1:200, 1:300, 1:400 or 1:500.

In one embodiment, a crosslinking agent is used to connect a targeting material to the surface of the drug composition.

In one embodiment, the targeting material is a monoclonal antibody Trastuzumab.

In one embodiment, the concentration of Trastuzumab includes 1 μg/mL, 2 μg/mL, or 3 μg/mL.

### BRIEF DESCRIPTION OF THE DRAWINGS

FIG. 1 shows a chemical structure of Carboxymethyl-Hexanoyl Chitosan (CHC) used in the present invention.

FIG. 2 shows SEM and TEM micrographs of morphologies of various drug compositions.

FIG. 3A shows release curves of free ganetespib and ganetespib encapsulated inside CHC nanocarriers (CHC/ganetespib).

FIG. 3B shows portions of the release curves in FIG. 3A, which appear in the time intervals from 0 to 15 hours.

FIG. 3C shows release curves of free curcumin and curcumin encapsulated inside CHC nanocarriers (CHC/curcumin).

FIG. 3D shows the release curves in FIG. 3C, wherein the scale of the accumulated release rates is adjusted.

FIG. 4A shows the results of cytotoxicity, wherein SK-BR-3cells are treated for 24 hours in different ratios of concentrations of free ganetespib and free curcumin.

FIG. 4B shows the results of cytotoxicity, wherein SK-BR-3cells are treated for 24 hours in different ratios of concentrations of ganetespib and curcumin encapsulated inside CHC nanocarriers.

FIG. 5 shows the histograms of cell survival rates of SK-BR-3cells, wherein SK-BR-3cells are treated for 48 hours in drug compositions containing different ratios of drugs.

FIG. 6A shows curves of body weights of Balb/c female nude mice within 2 weeks, wherein SK-BR-3cells are zenotransplanted into 7-week-old mice, and different drug compositions are used to treat the mice for 2 weeks.

FIG. 6B shows curves of tumor size of Balb/c female nude mice within 2 weeks, wherein SK-BR-3cells are zenotransplanted into 7-week-old mice, and different drug compositions are used to treat the mice for 2 weeks.

FIG. 6C shows the histograms of the tumor inhibition ratio of the drug-therapy groups in comparison with the control group using PBS.

### DETAILED DESCRIPTION OF THE INVENTION

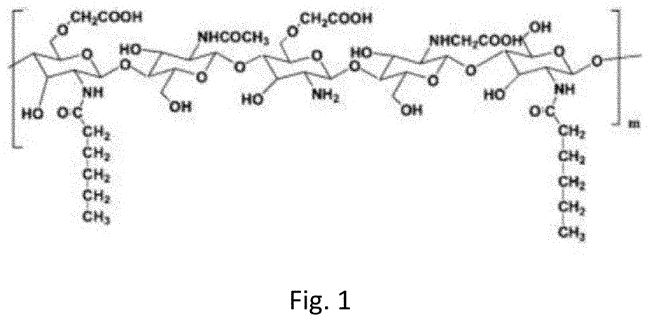

Below, FIGS. 1-6C are used to illustrate the embodiments of the present invention. However, it should be understood: these drawings and embodiments are only to exemplify the present invention but not to limit the scope of the present invention.

The present invention provides a drug composition for treating breast cancer, which comprises a nanocarrier, which is assembled with Carboxymethyl-Hexanoyl Chitosan (CHC); at least one heat shock protein 90 (HSP90) inhibitor; and at least one hydrophobic drug, wherein the HSP90 inhibitor and the hydrophobic drug are encapsulated inside the nanocarrier. The present invention also provides a method for manufacturing a drug composition for treating breast cancer, which comprises steps: dispersing Carboxymethyl-Hexanoyl Chitosan (CHC), at least one heat shock protein 90 (HSP90) inhibitor and at least one hydrophobic drug in a solvent to form a mixture solution; placing the mixture solution at a lower temperature and agitating the mixture solution for 20-24 hours to form the drug composition, wherein the HSP90 inhibitor and the hydrophobic drug are encapsulated inside the nanocarrier, which is assembled with Carboxymethyl-Hexanoyl Chitosan (CHC). In one embodiment, the drug composition is in form of a plurality of particles whose diameters are within a range of 200-500 nm.

Below are described in detail the methods of preparing the materials used in one embodiment.

### Amphiphilic Carboxymethyl-Hexanoyl Chitosan (CHC):

Chitosan is modified to generate amphiphilic chitosan. The amphiphilic chitosan is synthesized with a hydrophilic carboxymethyl substituent and a hydrophobic hexanoyl substituent. The chemical structure is shown in FIG. 1. The amphiphilic feature enables CHC to self-assemble into a nanocarrier, wherein the hydrophilic portion thereof forms the shell, and the hydrophobic portion thereof forms the core, whereby to increase the solubility of the hydrophobic drug and protect the drugs against the damage from the environment. According to the enhanced permeability and retention effect (EPR), the nanocarrier of CHC can pass through the gap between endothelial cells (200 nm-1.2 μm) and accumulates in cancer tissue. The carboxyl group in the CHC molecular chain may be modified with an antibody or a protein to provide the CHC nanoparticles with targeting ability. Thereby, the CHC nanoparticles can target cancer cells to perform treatment. Especially, the self-assembly CHC nanocarrier is biodegradable. CHC has an appropriate size, which prevents it from being filtered out by kidneys and makes it biodegradable by lysozyme.

### The Synthesis Method:

Take 10 g chitosan (purchased from CHARMING & BEAUTY Inc.) into a bottle. Add 100 mL isopropanol into the bottle, and agitate the mixture at a temperature of 25° C. for 30 minutes. Take 25 mL 13.3N sodium hydroxide aqueous solution, and divide the solution into 5 portions. The 5 portions of solution are added into the bottle in sequence at 5 timings, and two adjacent timings are separated by 5 minutes. Agitate the liquid in the bottle for 30 minutes. Divide 50 g chloroacetic acid into 5 portions. The 5 portions of chloroacetic acid are added into the bottle at 5 different timings within 5 minutes. Heat the bottle to a temperature of 60° C. for 4 hours. Add 1000 mL mixture solution of water and methyl alcohol (water:methyl alcohol=1:9 (v/v)). Use vacuum filtration to purify the product. Place the product in an oven, and dry the product at a temperature of 50° C. for 24 hours to obtain a white powder of N,O-carboxymethyl chitosan (NOCC). Take 4 g NOCC into a 250 mL reaction bottle. Add 100 mL pure water (ddH₂O) into the reaction bottle. Agitate the mixture solution in the reaction bottle for one day to make NOCC completely dissolve in water. Add 100 mL methyl alcohol to the reaction bottle, and make the methyl alcohol mix with the solution uniformly. Add 2.8 mL hexanoyl anhydride to the reaction bottle, and let reaction take place at a temperature of 25° C. for 12 hours. After reaction, use a dialysis bag to collect the solution. Dialyze the solution with a solution where pure water:ethanol=1:4 (v/v)) for one day, and then dialyze the solution with pure ethanol for one day. Collect the product, and dry the product at a temperature of 50° C. to obtain CHC powder.

In one embodiment, the HSP 90 inhibitor includes ganetespib, and the hydrophobic drug includes curcumin. The details thereof are described below.

### Ganetespib:

The second-generation HSP90 inhibitor is used to treat non-small cell lung cancer, breast cancer and prostate cancer. HSP90 is a molecular chaperone protein, which can modify the functions of proteins (such as EGFR, HER2, CDK4, etc.) via ubiquitylation (such as folding, maturation and stabilization). HSP90 over-expresses in cancer cells to support the growth, reproduction, anti-apoptosis and metastasis of cancer cells. Ganetespib is a resorcinol compound, able to competitively bind with the ATP binding domain of the N terminal of HSP90. Ganetespib is free of benzoquinone rings and thus has low dose-dependent hepatotoxicity. Ganetespib presents effective and persistent anti-cancer functions in in-vivo and in-vitro experiments. Ganetespib is often used to treat non-small cell lung cancer. Many experiments show that the HER2 over-expression breast cancer (HER2+) is very sensitive to the HSP90 inhibitor. The HSP90 inhibitor further have tremendous potential in treating triple negative breast cancer (TNBC).

### Curcumin:

Curcumin is a polyphenol compound extracted from the rhizome of curcuma longa. Curcumin has many medical effects and may function as anti-oxidants, antiviral drugs, antiinflammatory drugs and anticancer drugs. Curcumin has anti-proliferation function in many cancers. Curcumin is an inhibitor of the transcription factor NF-κB and the downstream gene products, including ct-myc, Bcl-2, COX-2, NOS, cyclin D1, TNF-α, interleukins, and MMP-9. In proliferation of breast cancer cells, NF-κB can modify more than 500 different genes and controls the expressions of the proteins participating in cellular signaling pathways. Thus, NF-κB may lead to cancer and inflammation. Curcumin can influence the proliferation ability and invasion ability of breast cancer cells via undertaking the negative regulation of the gene expressions induced by NF-κB. Curcumin is also a target of the human epidermal growth factor receptor 2 (HER2) that influences proliferation of breast cancer. Curcumin can inhibit breast cancer cells via inhibiting HER2-TK. Curcumin is promising in cancer treatment. However, insufficient bioavailability and low aqueous solubility impairs the development of curcumin in clinic. After entering human bodies, curcumin is quickly metabolized. The aqueous solubility of curcumin is very low, especially in an acidic environment and a neutral environment. Although curcumin can dissolve in a basic environment, it also fast decompose in a basic environment with the half-life thereof only few minutes. Besides, curcumin may suffer photocatalytic degradation in organic solvents. These properties limit the bioavailability of curcumin Further, curcumin may cause some side-effects, such as sickness, diarrhea, headache, and yellow stool.

In order to verify the effect of the drug composition where two drugs having different effects are encapsulated according to one embodiment of the present invention , three drug compositions are used in experiments, including a drug composition CHC/GAN where ganetespib (GAN) is encapsulated in carboxymethyl-hexanoyl chitosan (CHC); a drug composition CHC/CCM where curcumin (CCM) is encapsulated in carboxymethyl-hexanoyl chitosan (CHC); a drug composition CHC/GAN-CCM where ganetespib (GAN) and curcumin (CCM) are encapsulated in carboxymethyl-hexanoyl chitosan (CHC).

### Preparation of the Drug Composition CHC/GAN and the Drug Composition CHC/CCM:

Dissolve 5 mg ganetespib (GAN) in 250 μL dimethyl sulfoxide (DMSO) to form a 20 mg/mL reserve solution. Respectively dilute the reserve solution to a 1 mg/mL solution and a 100 μg/mL solution. Store the GAN reserve solution in a refrigerator at a temperature of −80° C. Dissolve 100 mg curcumin to form a 5 mg/mL reserve solution. Mix 50 μL GAN solution (the concentration in DMSO is 1 mg/mL) and 10% 400-PEG with 0.5 mg CHC powder in 1 mL ddH₂O for preparing the drug composition CHC/GAN. Mix 80 μL CCM solution (the concentration in DMSO is 5 mg/mL) and 10% 400-PEG with 0.5 mg CHC powder in 1 mL ddH₂O for preparing the drug composition CHC/CCM. Use a magnetic stirring apparatus to stir the solutions at a temperature of 4° C. for 24 hours in a darkroom to make the solutions self-assemble into the desired drug compositions where carriers encapsulate the drugs.

### Preparation of the Drug Composition CHC/GAN-CCM:

In order to find an ideal ratio of the concentrations of the drugs, GAN and CCM are prepared in different ratios of the concentrations: GAN:CCM=1:200, 1:300, 1:400 and 1:500, for in-vitro cytotoxicity experiments. The drug compositions CHC/GAN-CCM of the abovementioned ratios of concentrations are prepared via mixing 10 μL GAN (the concentration in DMSO is 100 μg/mL), 40, 60, 80, and 100 μL CCM (the concentration in DMSO is 5 mg/mL) and 10% 400-PEG in 1 mL ddH₂O. Use a magnetic stirring apparatus to stir all the solutions at a temperature of 4° C. for 20-24 hours in a darkroom to make the solutions self-assemble into the desired drug compositions where carriers encapsulate the drugs.

In one embodiment, the surface of the drug composition is connected with a targeting material. The targeting material is selected from a group including antibodies, peptides, and proteins.

In one embodiment, the antibodies include a monoclonal antibody Trastuzumab. Trastuzumab is a recombinant monoclonal antibody able to act on HER2. Trastuzumab is the first HER2 targeting breast cancer drug approved by FDA.

Modifying the Nano-Drug Composition with Trastuzumab:

Firstly, prepare the nano-drug composition CHC/GAN-CCM. Add 1 μL, 2 μL, and 3 μL Trastuzumab (the concentration thereof is 1 mg/mL in ddH₂O) to the solution of the nano-drug composition CHC/GAN-CCM. Agitate the solutions at a temperature of 4° C. for one hour. Add 50 μL EDC crosslinking agent (the concentration thereof is 1 mg/mL in ddH₂O). Agitate the solutions at a temperature of 4° C. for 4 hours to form amide bonds.

In one embodiment, ganetespib is used as the HSP90 inhibitor; curcumin is used as the hydrophobic drug; Trastuzumab is used as a targeting material. The experiments and results thereof are described below.

### Encapsulation Efficiency (EE):

1. Calibration line: prepare ganetespib solutions and curcumin solutions in various concentrations; dilute the solutions with methanol; detect the absorption intensities at 299 nm and 435 nm; use the results to plot the curves of the relationships of the concentrations and the absorption intensities as the calibration lines.
2. Encapsulation detection: agitate the solutions for 20-24 hours; centrifuge the solutions at a temperature of 4° C. and a speed of 10000 rpms for 10 minutes; take the supernatant liquids, and dilute the supernatant liquids with methanol; detect the light absorptions of the diluted supernatant liquids, and compare the light absorptions with the calibration lines to learn the concentrations of non-encapsulated drugs; substitute the concentrations of non-encapsulated drugs into the following equation to learn the encapsulation efficiency.

$$EE ( \% ) = ( 1 - \frac{concentrations of non \text{-} encapsulated drugs}{total concentration of drugs} )$$

The results are shown in Table.1 and Table.2. The encapsulation efficiencies of ganetespib of CHC/GAN, CHC/GAN-CCM and CHC/GAN-CCM@trastuzumab are respectively 62.2%, 37.0%, and 27.8%. The encapsulation efficiencies of curcumin of CHC/CCM, CHC/GAN-CCM and CHC/GAN-CCM@trastuzumab are respectively 81.0%, 77.6%, and 73.5%. The concentration of ganetespib used in the dual-drug composition is lower than that used in the single-drug composition. Therefore, the encapsulation efficiency of ganetespib in CHC/GAN-CCM significantly decreases. In self-assembly of CHC, the hydrophilic portion will form the shell, and the hydrophobic portion will form the core. Curcumin is highly hydrophobic. Therefore, curcumin has high encapsulation efficiencies in the single-drug composition and the dual-drug composition.

TABLE 1
encapsulation efficiency of ganetespib in CHC/GAN,
CHC/GAN-CCM and CHC/GAN-CCM@trastuzumab

| Sample | EE | STDEV | concentration |
|---|---|---|---|
| CHC/GAN | 62.2% | 4.63% | 50 μg/mL |
| CHC/GAN-CCM | 37.0% | 3.64% | 1 μg/mL |
| CHC/GAN-CCM@trastuzumab | 27.8% | 4.17% | 1 μg/mL |

TABLE 2
encapsulation efficiency of curcumin in CHC/CCM,
CHC/GAN-CCM and CHC/GAN-CCM@trastuzumab

| Sample | EE | STDEV | concentration |
|---|---|---|---|
| CHC/CCM | 81.0% | 2.52% | 400 μg/mL |
| CHC/GAN-CCM | 77.6% | 4.76% | 300 μg/mL |
| CHC/GAN-CCM@trastuzumab | 73.5% | 1.22% | 300 μg/mL |

In the present invention, dynamic light scattering (DLS) and zeta potential is used to measure the particle sizes and surface potentials of the drug compositions. The results are shown in Table.3. After the CHC carrier have encapsulated the drugs and connected with the antibody, DLS can detect that the particle diameter is relatively increased. Therefore, DLS can verify whether the antibody is successfully connected to the surface of the drug composition. The zeta potential method is used to detect the surface potentials of the drug compositions. It is found: the surface of CHC-encapsulated drug composition has positive charges in water solutions.

TABLE 3
results of measuring particle diameters and surface potentials

| | Particle | |
|---|---|---|
| | diameter | Zeta potential |
| Sample | (nm) | (mV) |
| CHC | 238.8 ± 5.86 | 24.26 ± 0.48 |
| CHC/GAN | 286.6 ± 1.51 | 24.03 ± 0.31 |
| CHC/CCM | 366.8 ± 7.28 | 21.14 ± 0.41 |
| CHC/GAN-CCM | 418.3 ± 5.34 | 20.82 ± 0.80 |
| CHC/GAN-CCM@trastuzumab | 504.2 ± 11.28 | 18.17 ± 0.50 |

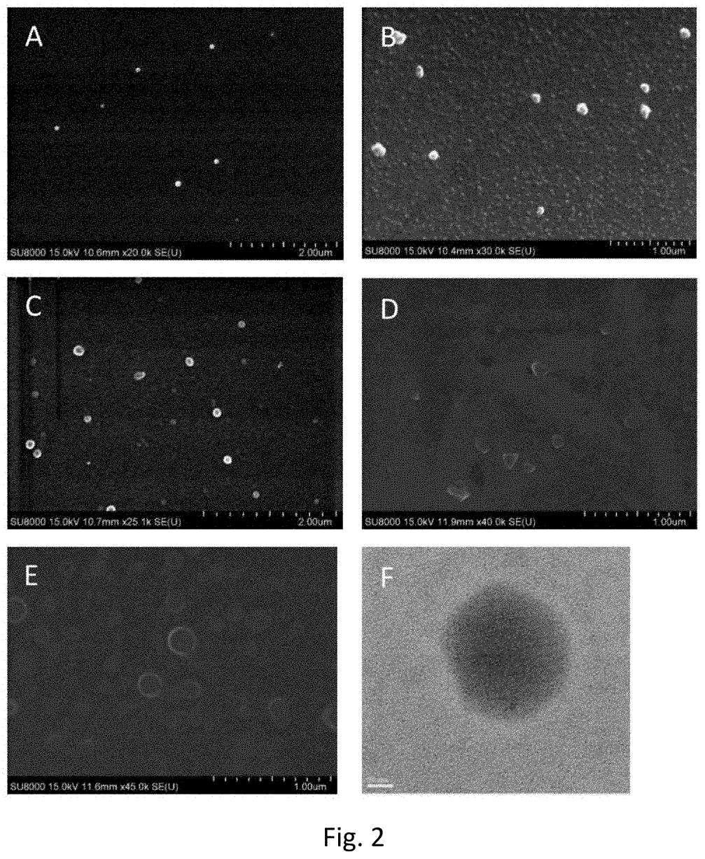

In the present invention, the scanning electron microscope (SEM) and the transmission electron microscope (TEM) are used to observe the morphologies of the drug compositions, and the photographs are shown in FIG. 2. Photo A shows the SEM-based morphology of the CHC carriers that do not encapsulate drugs thereinside. Photo B shows the SEM-based morphology of the drug composition CHC/GAN where CHC carriers encapsulate ganetespib. Photo C shows the SEM-based morphology of the drug composition CHC/CCM where CHC carriers encapsulate curcumin Photo D shows the SEM-based morphology of the drug composition CHC/GAN-CCM where CHC carriers encapsulate ganetespib and curcumin. Photo E shows the SEM-based morphology of the drug composition where CHC carriers encapsulate ganetespib and curcumin and antibodies are grafted on the surface of CHC carriers. Photo F shows the TEM-based morphology of the drug composition CHC/GAN-CCM where carriers encapsulate ganetespib and curcumin

### Release of Drugs:

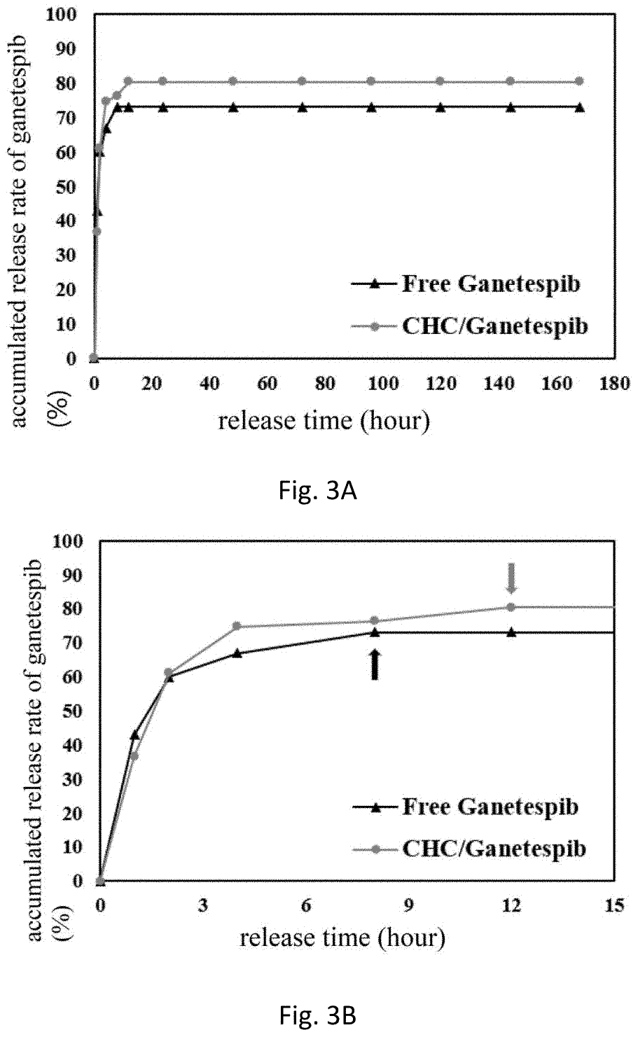

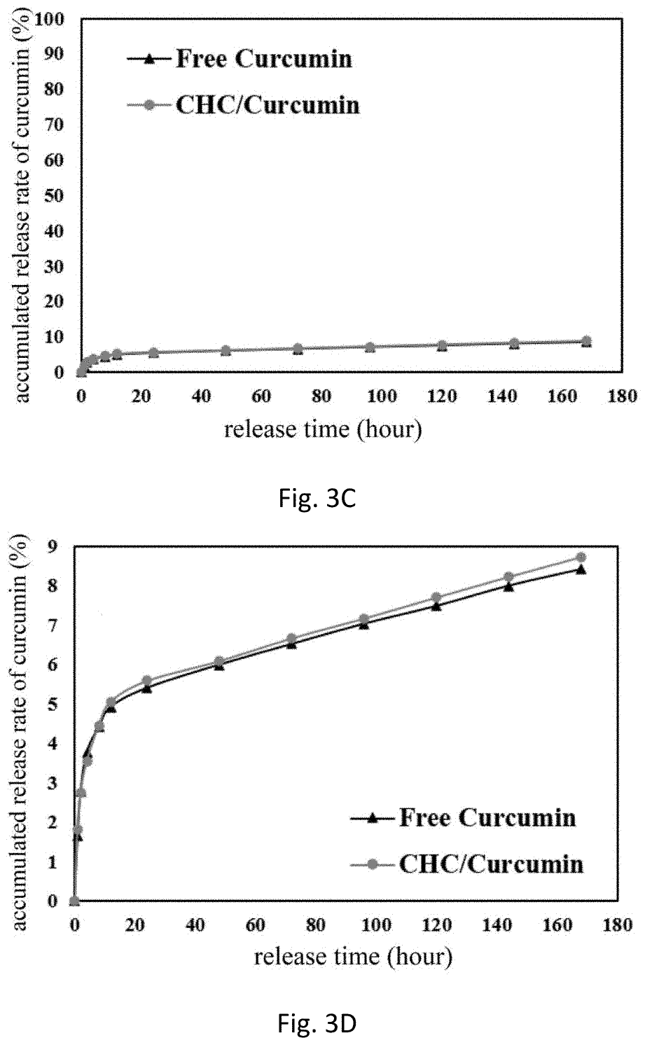

FIG. 3 shows the release curves of ganetespib of CHC/GAN and curcumin of CHC/CCM, which are measured in a pH 7.4 PBS buffer solution for 168 hours at 37° C. FIG. 3A and FIG. 3B show the release curves of ganetespib. FIG. 3C and FIG. 3D show the release curves of curcumin For free ganetespib, the dialysis bag stops release after 8 hours, and about 73% ganetespib is released. For ganetespib in the drug composition, the dialysis bag continues release until as long as 12 hours has elapsed, and about 80% ganetespib is released. As indicated by the arrows in FIG. 3B, CHC/GAN where ganetespib is encapsulated by CHC releases ganetespib more slowly and has higher content of ganetespib. It is because CHC protects ganetespib from being damaged by the environment and makes ganetespib release slowly. For free curcumin, although the dialysis bag have continued releasing curcumin for as long as 168 hours, the accumulated released curcumin is only about 8%. The slow release rate of free curcumin is owing to low aqueous solubility of curcumin Low aqueous solubility of curcumin also leads to low bioavailability of curcumin. The release rate of the curcumin in CHC/CCM is about the same as the free curcumin. The accumulated released curcumin of the curcumin in CHC/CCM is about 9%, slightly higher than that of the free curcumin. However, it can be seen in FIG. 3D: the difference therebetween increases with time.

### Cytotoxicity Test:

Culture SK-BR-3 cells in a 24-well culture plate, and each well has 10⁴ cells. After cells have attached to the culture plate for 20-24 hours, remove the culture liquid, and rinse the cells with PBS. Use Dulbecco's Modified Eagle Medium (DMEM) to dilute the drug compositions GAN, CCM, CHC/GAN, CHC/CCM, and CHC/GAN-CCM. Add the diluted drug compositions to the cells, and co-culture each drug composition and the cells for 48 hours. Remove DMEM, and flush the product of co-culture with PBS. Add MTS and DMEM (1:5 (v/v)) into each well of the cell culture plate, and culture them for 2-4 hours. Use a microplate reader to measure the absorptivity of the light having a wavelength of 490 nm, whereby to estimate the cell survival rates. The cell survival rate can be used to determine the half inhibitory concentration (IC₅₀) and the combination index (CI) of two drugs. The results are shown in Table.4. CHC/GAN and CHC/CCM can more effectively kill cells than the drugs in form of free molecules. It indicates that CHC nanocarriers can transfer drugs more efficiently. Therefore, the drug composition carried by CHC nanoparticles can more effectively treat breast cancer in smaller dosage. Especially, while ganetespib is carried by CHC nanocarriers, the treating effect may be increased 60-70 times. The results indicate that the drugs encapsulated by carriers are more cytotoxic than free drugs. The results also indicate that the effective concentration of the drug composition of the present invention may be lower than that of the conventional drug composition. Therefore, the present invention can decrease the side-effects of drugs.

TABLE 4
the effects of free ganetespib and curcumin and the CHC
encapsulated drug compositions on IC₅₀ of SK-BR-3 cells

| Drug | IC₅₀ |
|---|---|
| GAN | 1.88 ng/mL |
| CHC/GAN | 0.028 ng/mL |
| CCM | 12.02 μg/mL |
| CHC/CCM | 8.29 μg/mL |

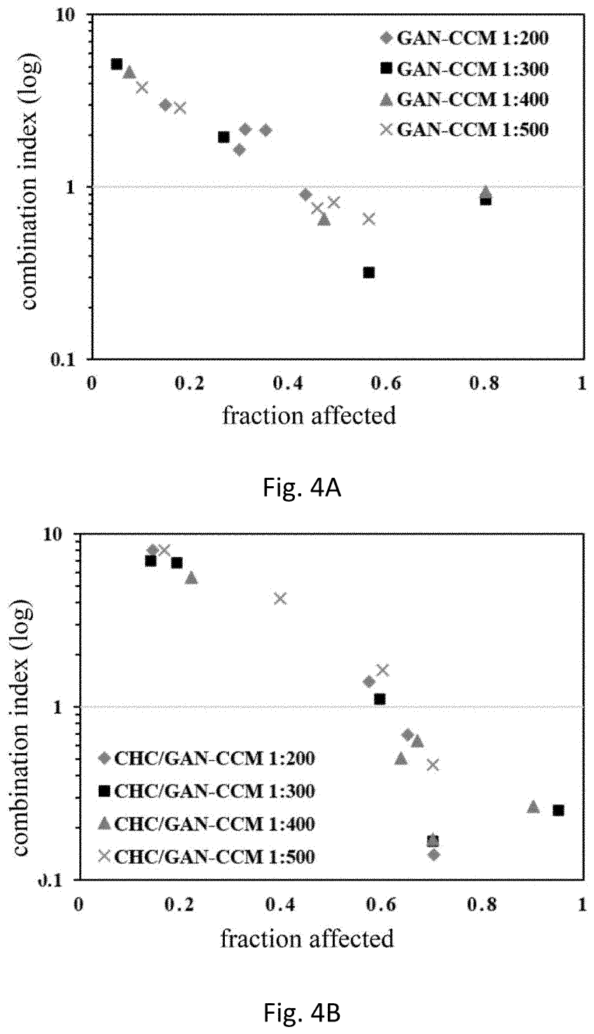

The so-called synergetic effect means that two drugs having different working mechanisms are used in a specific ratio to complementarily enhance the functions of the two drugs. In the present invention, experiments analyze four combinations of free drugs in four different ratios (GAN:CCM=1:200, 1:300, 1:400, 1:500) and the drugs encapsulated by CHC nanocarriers. According to the theory of Chou-Talalay, while the combination index (CI) is smaller than 1, the synergetic effect will take place in two drugs; while the combination index (CI) is greater than or equal to 1, the additive promotion and antagonistic effect will take place. The relationships of the combination index (CI) and the fraction affected (FA) are shown in FIG. 4A and FIG. 4B. A higher FA means a lower cell survival rate. Free dual-drug compositions have lower FA and are very likely to have the antagonistic effect. However, the CHC-encapsulated dual-drug compositions present the synergetic effect and higher FA, especially the CHC/GAN-CCM drug composition where GAN:CCM=1:300. As shown in FIG. 4B, the case where GAN:CCM=1:300 is the preferred embodiment of synergetic therapy.

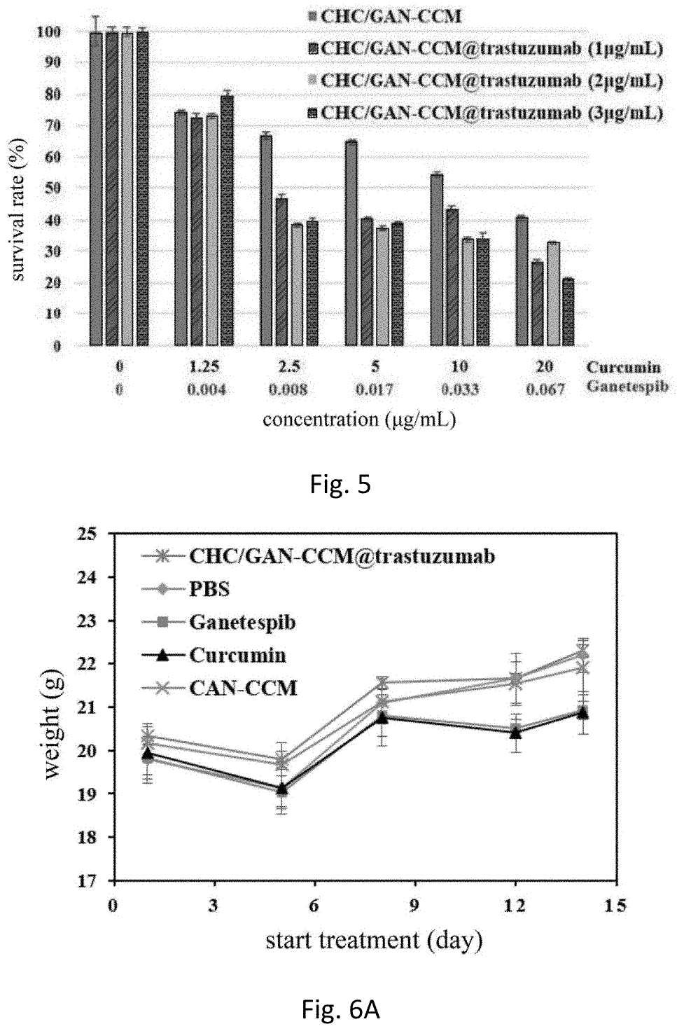

According to the experiments of estimating the CI value, the embodiment where GAN:CCM=1:300 is used in the following experiment. The surface of CHC/GAN-CCM is modified with the monoclonal antibody Trastuzumab via the EDC crosslinking agent. The concentrations of the monoclonal antibody Trastuzumab for surface modification are respectively 1 μg/mL, 2 μg/mL, and 3 μg/mL. CHC/GAN-CCM and CHC/GAN-CCM@Trastuzumab of the abovementioned three concentrations are used to treat SK-BR-3 cells for 48 hours, and calculate the cell survival rates to compare the treating effects. As shown in FIG. 5, the nanocompositions modified with Trastuzumab outperform the nanocomposition not modified with Trastuzumab. The nanocompositions modified with 3 μg/mL Trastuzumab has the highest cytotoxicity, especially for the case having a higher GAN/CCM concentrations.

### Animal Experiments:

Divide 20 7-week-old female Balb/c nude mice into 5 group. Inject 1×10⁷ SK-BR-3 cells (100 μL of PBS where the concentration is 10⁸ cells/mL) into the right flank region of the mice. The volume of the cancer is calculated according to the formula: V=(L×W×H)/2, wherein L=length (the longest dimension); W=width (the dimension vertical to the length and on the same plane where the length exists); H=height (the distance between the external boundary of the cancer and the body of the mouse). After the volume of the cancer has reached 70-80 mm³, use PBS (the control group), free ganetespib, free curcumin, the combination of free ganetespib and free curcumin, CHC/GAN-CCM@Trastuzumab to treat the 5 groups of mice. Inject the abovementioned 5 compositions into the bodies of the mice through the tail veins once per week, and the injection is undertaken for two successive weeks. The dosage of each injection is 100 ml/20 kg. The dosages of ganetespib and curcumin are respectively 0.1 mg/kg and 30 mg/kg (the ratio of ganetespib and curcumin=1:300). The concentration of CHC is 0.5 wt %, and the concentration of Trastuzumab is 3 μg/mL. After the first injection, the body weights of the mice and the sizes of the tumors are measured twice per week.

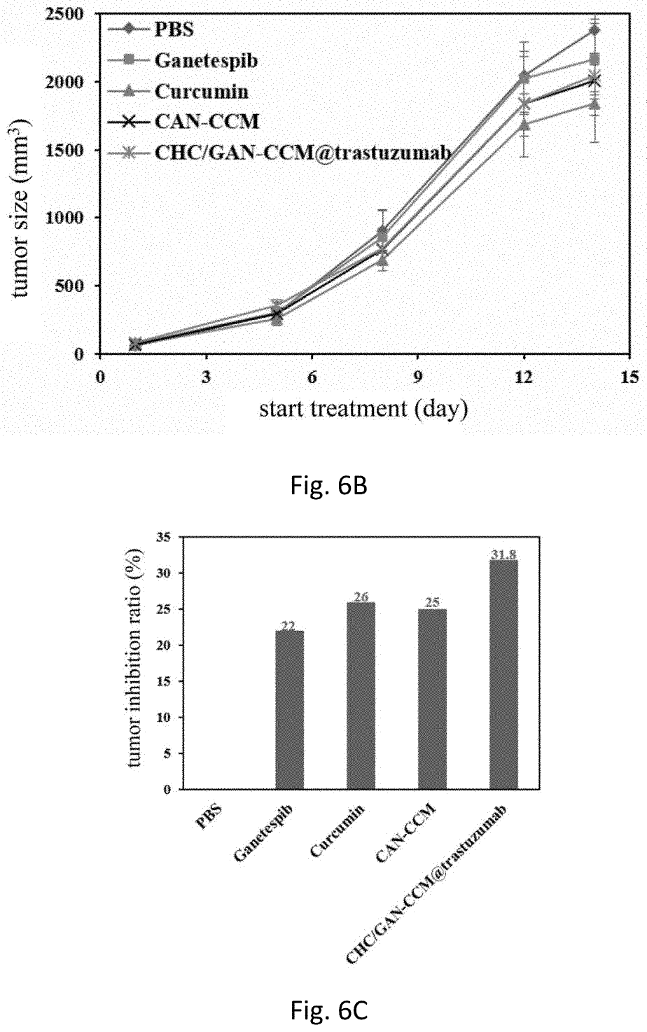

As shown in FIG. 6A, the body weights of all the mice are maintained over 20 g, and the physiological states of all the mice are maintained normal. It indicates that the dosages (ganetespib 0.1 mg/kg, curcumin 30 mg/kg) are suitable for cancer therapy and almost nontoxic for the whole body. As shown in FIG. 6B, the sizes of the tumors of the drug-therapy groups are all smaller than 2000 mm³; the sizes of the tumors of the control group are about 2300 mm³. It indicates that ganetespib and curcumin can effectively inhibit tumors in vivo. As shown in FIG. 6C, compared with the control group, the tumor inhibition ratio of the drug-therapy groups are all higher than 20%, especially the CHC/GAN-CCM@Trastuzumab group, which has the tumor inhibition ratio of as high as 31.8%. Therefore, the embodiment where the carriers are modified with antibodies not only presents a targeting feature but also presents a synergetic effect in a specified drug concentration ratio to resist malignant cancers.

In conclusion, the present invention provides an amphiphilic self-assembly CHC nanocarrier features bioavailability and low toxicity. In the in-vitro experiments of SK-BR-3 breast cancer cells, the IC₅₀ of the drug compositions of the present invention is far lower than the IC₅₀ of the drugs in form of free molecules. It indicates that the effective concentrations of the drug compositions of the present invention may be lower than that of the drugs in form of free molecules. Thus, the side-effects of drugs are decreased. Further, the dual-drug composition in a specified concentration ratio presents a synergetic effect in in-vivo and in-vitro experiments. Thus, the present invention can avoid multidrug resistance. Furthermore, CHC not only may increase the solubility of hydrophobic drugs (such as curcumin) but also can protect them from be degraded by the environment, whereby the bioavailability thereof is increased. Moreover, the surface of the CHC nanocarrier may be modified with targeting materials (such as the monoclonal antibody Trastuzumab), whereby the drug composition of the present invention can target the HER2-positive cancer cells and treat cancer more effectively.

The embodiments have been described above to demonstrate the principles of the present invention and enable the persons skilled in the art to understand, make, and use the present invention. However, these embodiments are only to exemplify the present invention but not to limit the scope of the present invention. The technical thought and scope of the present invention is defined by the claims stated below and the equivalents thereof. Any simplification, substitution, combination, modification or variation according to the principle, spirit or embodiment of the present invention is to be also included by the scope of the present invention.

What is claimed is:

1. A drug composition for treating breast cancer, comprising:
a nanocarrier, wherein Carboxymethyl-Hexanoyl Chitosan (CHC) self-assemble to form said nanocarrier;
at least one Heat Shock Protein 90 (HSP90) inhibitor; and
at least one hydrophobic drug;
wherein said HSP90 inhibitor and said hydrophobic drug are encapsulated inside said nanocarrier; and
wherein said HSP90 inhibitor includes ganetespib and said hydrophobic drug includes curcumin.

2. The drug composition according to claim 1, wherein said drug composition includes a plurality of particles whose diameter ranges from 200-500 nm.

3. The drug composition according to claim 1, wherein a surface of said drug composition is connected with a targeting material.

4. The drug composition according to claim 3, wherein said targeting material includes a monoclonal antibody Trastuzumab.

5. The drug composition according to claim 4, wherein said breast cancer is an HER2 overexpression breast cancer.

6. A method for manufacturing a drug composition for treating breast cancer, comprising steps:
dispersing Carboxymethyl-Hexanoyl Chitosan (CHC), at least one Heat Shock Protein 90 (HSP90) inhibitor and at least one hydrophobic drug in a solvent to form a mixture solution; and
agitating said mixture solution at a low temperature for 20-24 hours to form said drug composition;
wherein said HSP inhibitor and said hydrophobic drug of said drug composition are encapsulated inside a nanocarrier, which is formed by self-assembly of CHC, and
wherein said HSP90 inhibitor includes ganetespib and said hydrophobic drug includes curcumin.

7. The method according to claim 6, wherein ratios of concentrations of said ganetespib and said curcumin include 1:200, 1:300, 1:400 and 1:500.

8. The method according to claim 6 further comprising a step: using a crosslinking agent to connect a targeting material to a surface of said drug composition.

9. The method according to claim 8, wherein said targeting material includes a monoclonal antibody Trastuzumab.

10. The method according to claim 9, wherein concentrations of said monoclonal antibody Trastuzumab include 1 μg/mL, 2 μg/mL and 3 μg/mL.
